(19) United States
(12) Patent Application Publication
(10) Pub. No.: US 2015/0005814 A1
(43) Pub. Date: Jan. 1, 2015

(54) Tweezers

(71) Applicant: Zwilling J.A. Henckels AG, Solingen (DE)

(72) Inventor: Paul Boldt, Solingen (DE)

(21) Appl. No.: 14/317,805

(22) Filed: Jun. 27, 2014

(30) Foreign Application Priority Data
Jun. 28, 2013 (DE) 10 2013 010 785.7

(51) Int. Cl. A61B 17/30; B22D 17/00

(52) U.S. Cl. CPC A61B 17/30; B22D 17/00; C22C 45/10
USPC 606/210; 164/113; 164/6

(57) ABSTRACT

Tweezers, in particular, cosmetic tweezers, include two legs extending in longitudinal direction. The two legs have connecting ends and free ends and are connected to one another at their connecting ends. The free ends form inner jaw surfaces shaped to hold an object therebetween. Both of the legs and the connecting area are of a metallic glass or of an amorphous metal in one piece.

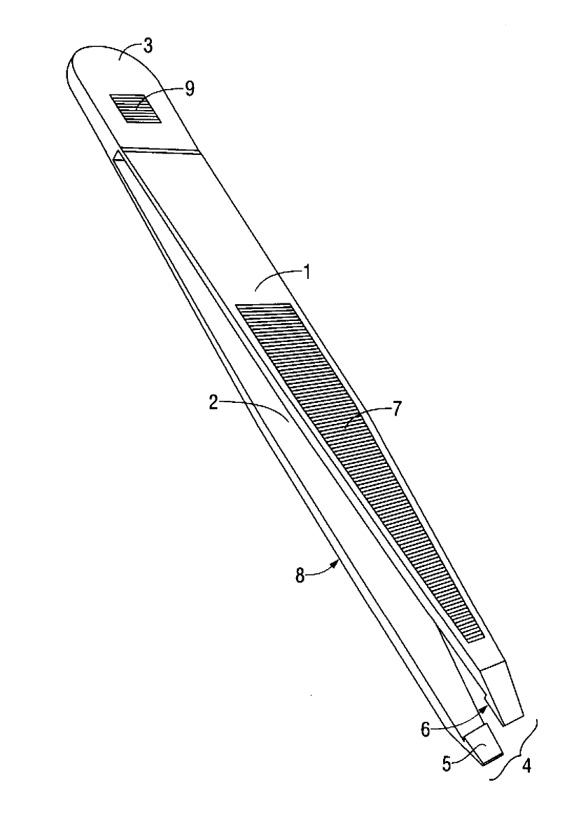

### CROSS-REFERENCE TO RELATED APPLICATIONS

This application claims the priority, under 35 U.S.C. §§119 and 371, of German patent application No. 10 2013 010 785.7, filed Jun. 28, 2013; the prior application is herewith incorporated by reference in its entirety.

### STATEMENT REGARDING FEDERALLY SPONSORED RESEARCH OR DEVELOPMENT

Not Applicable

### FIELD OF THE INVENTION

The present invention lies in the field of implements. The invention relates to tweezers, in particular cosmetic tweezers, comprising two legs extending in longitudinal direction, which are connected to one another with their one ends and the free ends of which form inner jaw surfaces that are able to hold something between both jaw surfaces.

### BACKGROUND OF THE INVENTION

It is known to produce tweezers from two metal profiles, which are joined at one end. In addition to the joining, several additional operations are also required, such as the production of ribbing or ribs at the inner jaw surfaces and ribbing at the outer handle surfaces. Also required is the tapering of material>calanders to effect the spring force and grinding, blasting, etc., to bun the inner surface, and producing the step and the outer bevel of the jaw. Finally, further grinding operations are necessary.

Thus, a need exists to overcome the problems with the prior art systems, designs, and processes as discussed above.

### SUMMARY OF THE INVENTION

The invention provides tweezers that overcome the hereinafore-mentioned disadvantages of the heretofore-known devices of this general type and that provide such features with as few operating steps as possible. In particular, both legs, including the connecting area, are of a metallic glass or of an amorphous metal in one piece.

The invention relates to tweezers, in particular cosmetic tweezers, comprising two legs extending in longitudinal direction, which are connected to one another with their one ends and the free ends of which form inner jaw surfaces, so as to hold something between both jaw surfaces, wherein both legs including the connecting area consist of a metallic glass or of an amorphous metal in one piece.

In the case of such a one-piece production, the entire pair of tweezers with all of its parts and areas is produced in a single injection molding process, so that it is not necessary to subsequently fasten the legs of the tweezers to one another and extensive machine finishing operations are no longer necessary.

With the injection molding in one piece, the legs of the tweezers and the connection location have the final shape, so that subsequent profiling, ribbing, ribs are not necessary and so that even embellishments and the application of a logo already exist with the first operating process. A high degree of creative freedom, therefore, exists. A pleasing visual appearance can be attained, because more design and freedom/leeway/variances are at hand, in particular, due to certain designs of the surfaces, such as ergonomic shaping and/or ribbing.

The chosen material is highly elastic, free from corrosion, and has a high durability and a low wear/low abrasion. The production effort is extremely low. The number of production steps (stamping, punching, shaping, among others) is significantly reduced. Due to the high dimensional accuracy, no abrasive work or little abrasive work is required. If at all, grinding and/or polishing is only still required on the outside.

Due to the fact that a machine finishing is not required or is hardly required, a significant amount of material is saved.

In a method for producing tweezers of the above-mentioned type, it is preferable for the metallic glass or the amorphous metal to be injected into a mold, the cavity/cavities of which form both legs of the tweezers and the connecting area.

Preferably, the metallic glass or the amorphous metal of the tweezers contains zirconium as base.

With the foregoing and other objects in view, there is provided, in accordance with the invention, tweezers comprising two legs extending in a longitudinal direction and having first ends connected to one another at a connecting area and free ends opposite the first ends, the free ends forming inner jaw surfaces shaped to hold an object therebetween. The two legs and the connecting area are at least one of a one-piece metallic glass and a one-piece amorphous metal.

With the objects of the invention in view, there is also provided a method for producing tweezers, which comprises providing an injection mold having cavities shaping two legs extending in a longitudinal direction and having first ends connected to one another at a connecting area and free ends forming inner jaw surfaces shaped to hold an object therebetween, and injecting at least one of metallic glass and amorphous metal into the mold to form a one-piece tweezers having both of the two legs and the connecting area.

With the objects of the invention in view, there is also provided a method for producing tweezers, which comprises forming an injection mold with cavities in a shape of two legs extending in a longitudinal direction and having first ends connected to one another at a connecting area and free ends forming inner jaw surfaces shaped to hold an object therebetween, and injecting at least one of metallic glass and amorphous metal into the mold to form a one-piece tweezers having both of the two legs and the connecting area.

In accordance with another feature of the invention, the two legs and the connecting area comprise a one-piece zirconium-based metallic glass.

In accordance with a further feature of the invention, the two legs and the connecting area comprise a one-piece zirconium-based amorphous metal.

In accordance with an added feature of the invention, the two legs and the connecting area form a one-piece cosmetic tweezers.

In accordance with an additional mode of the invention, the two legs and the connecting area injected molded with the metallic glass by injection into a mold having cavities that form all of the two legs and the connecting area.

In accordance with yet another mode of the invention, the two legs and the connecting area are injected molded with the amorphous metal by injection into a mold having cavities that form all of the two legs and the connecting area.

In accordance with yet a further feature of the invention, the injecting step is carried out with zirconium-based metallic glass to form the two legs and the connecting area in one-piece.

In accordance with a concomitant feature of the invention, the injecting step is carried out with a zirconium-based amorphous metal to form the two legs and the connecting area in one-piece.

Although the invention is illustrated and described herein as embodied in tweezers, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Additional advantages and other features characteristic of the present invention will be set forth in the detailed description that follows and may be apparent from the detailed description or may be learned by practice of exemplary embodiments of the invention. Still other advantages of the invention may be realized by any of the instrumentalities, methods, or combinations particularly pointed out in the claims.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing FIGURES, in which like reference numerals are carried forward.

### BRIEF DESCRIPTION OF THE DRAWING

The accompanying FIGURE, which is not true to scale, and which, together with the detailed description below, is incorporated in and form part of the specification, serves to illustrate embodiments and to explain various principles and advantages all in accordance with the present invention. Advantages of embodiments of the present invention will be apparent from the following detailed description of the exemplary embodiments thereof, which description should be considered in conjunction with the accompanying drawings in which, the FIGURE is a perspective view of a tweezers.

### DETAILED DESCRIPTION OF THE INVENTION

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing FIGURES, in which like reference numerals are carried forward.

Alternate embodiments may be devised without departing from the spirit or the scope of the invention. Additionally, well-known elements of exemplary embodiments of the invention will not be described in detail or will be omitted so as not to obscure the relevant details of the invention.

Before the present invention is disclosed and described, it is to be understood that the terminology used herein is for the purpose of describing particular embodiments only and is not intended to be limiting. The terms “a” or “an”, as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

Described now are exemplary embodiments of the present invention. Referring now to the FIGURE of the drawing in detail, there is shown an exemplary embodiment of tweezers encompass a first leg 1 and a second leg 2, one end area of each of which is connected in one piece and in a positive manner to a connecting area 3 and the free ends 4 of which form jaw surfaces 5, 6. On the outer sides, the legs 1, 2 respectively encompass an outer handle surface 7, 8, which in an exemplary embodiment has ribbing or ribs. In an exemplary embodiment, a logo 9 is embossed on the outer side of the connecting area 3.

The entire pair of tweezers is of a metallic glass or of an amorphous metal in one piece and is injected into an injection mold in an injection molding tool. The one cavity (hollow space) of such an injection mold forms all of the parts of the tweezers including both legs 1, 2 and the connecting area 3 as well as all of the profiling, embellishments, ribbing, and ribs. A machine finishing of the tool is, therefore, no longer required. Thus, the finished tweezers are produced in a single production process from a material that is highly elastic and free from corrosion while having a high durability and, thus, minimal wear.

The metallic glass or the amorphous metal has zirconium as a base or is zirconium-based.

It is noted that various individual features of the inventive processes and devices may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing FIGURE or description. Further, where two or more reference numerals are used in the FIGURES or in the drawings, this should not be construed as being limited to only those embodiments or features, they are equally applicable to similar features or not a reference numeral is used or another reference numeral is omitted.

The phrase “at least one of A and B” is used herein and/or in the following claims, where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables.

The foregoing description and accompanying drawing illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. Tweezers, comprising:
two legs extending in a longitudinal direction and having:
first ends connected to one another at a connecting area; and
free ends opposite the first ends, the free ends forming inner jaw surfaces shaped to hold an object therebetween, the two legs and the connecting area being at least one of:
a one-piece metallic glass; and
a one-piece amorphous metal.

2. The tweezers according to claim 1, wherein the two legs and the connecting area comprise a one-piece zirconium-based metallic glass.

3. The tweezers according to claim 1, wherein the two legs and the connecting area comprise a one-piece zirconium-based amorphous metal.

4. The tweezers according to claim 1, wherein the two legs and the connecting area form a one-piece cosmetic tweezers.

5. A method for producing tweezers according to claim 1, wherein the two legs and the connecting area are injected molded with the metallic glass by injection into a mold having cavities that form all of the two legs and the connecting area.

6. A method for producing tweezers according to claim 1, wherein the two legs and the connecting area are injected molded with the amorphous metal by injection into a mold having cavities that form all of the two legs and the connecting area.

7. A method for producing tweezers, which comprises:
providing an injection mold having cavities shaping two legs extending in a longitudinal direction and having:
first ends connected to one another at a connecting area; and
free ends forming inner jaw surfaces shaped to hold an object therebetween; and
injecting at least one of metallic glass and amorphous metal into the mold to form a one-piece tweezers having both of the two legs and the connecting area.

8. The method according to claim 7, which further comprises carrying out the injecting step with zirconium-based metallic glass to form the two legs and the connecting area in one-piece.

9. The method according to claim 7, which further comprises carrying out the injecting step with a zirconium-based amorphous metal to form the two legs and the connecting area in one-piece.

10. The tweezers according to claim 7, wherein the two legs and the connecting area form a one-piece cosmetic tweezers.

11. A method for producing tweezers, which comprises:
forming an injection mold with cavities in a shape of two legs extending in a longitudinal direction and having:
first ends connected to one another at a connecting area; and
free ends forming inner jaw surfaces shaped to hold an object therebetween; and
injecting at least one of metallic glass and amorphous metal into the mold to form a one-piece tweezers having both of the two legs and the connecting area.

12. The method according to claim 11, which further comprises carrying out the injecting step with zirconium-based metallic glass to form the two legs and the connecting area in one-piece.

13. The method according to claim 11, which further comprises carrying out the injecting step with a zirconium-based amorphous metal to form the two legs and the connecting area in one-piece.

14. The tweezers according to claim 11, wherein the two legs and the connecting area form a one-piece cosmetic tweezers.
